(19) United States
(12) Patent Application Publication
(10) Pub. No.: US 2015/0005661 A1
(43) Pub. Date: Jan. 1, 2015

(54) METHOD AND PROCESS FOR REDUCING TINNITUS

(71) Applicant: Lloyd Trammell, Thousand Oaks, CA (US)

(72) Inventor: Lloyd Trammell, Thousand Oaks, CA (US)

(73) Assignee: Max Sound Corporation, La Jolla, CA (US)

(21) Appl. No.: 14/188,609

(22) Filed: Feb. 24, 2014

Related U.S. Application Data
(60) Provisional application No. 61/768,300, filed on Feb. 22, 2013.

(51) Int. Cl. A61B 5/12

(52) U.S. Cl. CPC A61B 5/128
USPC 600/559

(57) ABSTRACT

A process and system for reducing Tinnitus includes receiving an input audio sound by a user. The user listens to different tones (frequencies) of the received audio to determine both amplitude and frequency of the ringing or tinnitus tone in user's ears and provides tactile response on an input device (keyboard, etc.). The offending frequencies and amplitudes are identified based on the user's responses and a diagnosis from the data collected is produced and a tone map for each ear separately is created and the collection of tones and amplitudes are provided to the user to hear. User is tested for amplitude of these tones and the tones are increased until the audible ringing disappears as the phase of these tones will be shifted from zero to negative 180 degrees and as their level is increased, the ringing will decrease until it is substantially reduced.

### CROSS-REFERENCE TO RELATED PATENT APPLICATIONS

Embodiments of the present invention relate to U.S. Provisional Application Ser. No. 61/768,300, filed Feb. 22, 2013, entitled “PROCESS FOR REDUCING TINNITUS”, the contents of which are incorporated by reference herein and which is a basis for a claim of priority.

### BACKGROUND OF THE INVENTION

Tinnitus; from the Latin word tinnitus meaning “ringing” is the perception of sound within the human ear (ringing of the ears) when no actual sound is present. Despite the origin of the name, “ringing” is only one of many sounds the person may perceive.¹ ¹http://en.wikipedia.org/wiki/Tinnitus

Tinnitus is not a disease, but a condition that can result from a wide range of underlying causes. The most common cause is noise-induced hearing loss. Other causes include: neurological damage (multiple sclerosis), ear infections, oxidative stress, emotional stress, foreign objects in the ear, nasal allergies that prevent (or induce) fluid drain, wax build-up, and exposure to loud sounds. Withdrawal from benzodiazepines may cause tinnitus as well. Tinnitus may be an accompaniment of sensorineural hearing loss or congenital hearing loss, or it may be observed as a side effect of certain medications (ototoxic tinnitus)². ²See, n.1, above.

[Tinnitus is usually a subjective phenomenon, such that it cannot be objectively measured. The condition is often rated clinically on a simple scale from “slight” to “catastrophic” according to the difficulties it imposes, such as interference with sleep, quiet activities, and normal daily activities³. ³ See, n.1, above.

If there is an underlying cause, treating it may lead to improvements. Otherwise typically management involves talk therapy. As of 2013, there are no effective medications. It is common, affecting about 10-15% of people, most however tolerate it well with it being only a significant problem in 1-2% of people⁴. ⁴See, n.1, above.

Tinnitus can be perceived in one or both ears or in the head. It is usually described as a ringing noise, but in some patients, it takes the form of a high-pitched whining, electric buzzing, hissing, humming, tinging or whistling sound, or as ticking, clicking, roaring, “crickets” or “tree frogs” or “locusts (cicadas)”, tunes, songs, beeping, sizzling, sounds that slightly resemble human voices or even a pure steady tone like that heard during a hearing test, and in some cases, pressure changes from the interior ear. It has also been described as a “whooshing” sound because of acute muscle spasms, as of wind or waves. Tinnitus can be intermittent, or it can be continuous, in which case it can be the cause of great distress. In some individuals, the intensity can be changed by shoulder, head, tongue, jaw, or eye movements⁵. ⁵See, n.1, above.

Most people with tinnitus have some degree of hearing loss, in that they are often unable to hear clearly external sounds that occur within the same range of frequencies as their “phantom sounds”. This has led to the suggestion that one cause of tinnitus might be a homeostatic response of central dorsal cochlear nucleus auditory neurons that makes them hyperactive in compensation to auditory input loss⁶. ⁶See, n.1, above.

The sound perceived may range from a quiet background noise to one that can be heard even over loud external sounds. The specific type of tinnitus called pulsatile tinnitus is characterized by one hearing the sounds of one's own pulse or muscle contractions, which is typically a result of sounds that have been created from the movement of muscles near to one's ear, changes within the canal of one's ear or issues related to blood flow of the neck or face⁷. ⁷See, n.1, above.

Persistent tinnitus may cause irritability, fatigue, and on occasions, clinical depression and musical hallucinations. Tinnitus annoyance is more strongly associated with psychological symptoms than acoustic characteristics. Other psychological problems such as depression, anxiety, sleep disturbances and concentration difficulties are common in those with worse tinnitus⁸. ⁸See, n.1, above.

The basis of quantitatively measuring tinnitus relies on the brain's tendency to select out only the loudest sounds heard. Based on this tendency, the amplitude of a patient's tinnitus can be measured by playing sample sounds of known amplitude and asking the patient which they hear. The volume of the tinnitus will always be equal to or less than that of the sample noises heard by the patient. This method works very well to gauge objective tinnitus (see above). For example: if a patient has a pulsatile paraganglioma in their ear, they will not be able to hear the blood flow through the tumor when the sample noise is 5 decibels louder than the noise produced by the blood. As sound amplitude is gradually decreased, the tinnitus will become audible, and the level at which it does so provides an estimate of the amplitude of the objective tinnitus⁹. ⁹See, n.1, above.

Objective tinnitus, however, is quite uncommon. Often patients with pulsatile tumors will report other coexistent sounds, distinct from the pulsatile noise, which will persist even after their tumor has been removed. This is generally subjective tinnitus, which, unlike the objective form, cannot be tested by comparative methods. However, pulsatile tinnitus can be a symptom of intracranial vascular abnormalities, and should be evaluated for bruits by a medical professional with auscultation over the neck, eyes, and ears. If the exam reveals a bruit, imaging studies such as transcranial doppler (TCD) or magnetic resonance angiography (MRA) should be performed¹⁰. ¹⁰See, n.1, above.

Assessment of psychological processes related to tinnitus involves measurement of tinnitus severity and distress (i.e. nature and extent of tinnitus-related problems), measured subjectively by validated self-report tinnitus questionnaires. However, wide variability, inconsistencies and lack of consensus regarding assessment methodology is evidenced in the literature, limiting comparison of treatment effectiveness. Developed to guide diagnosis or classify severity, most tinnitus questionnaires have also been shown to be treatment-sensitive outcome measures¹¹. ¹¹See, n.1, above.

What is needed is an effective and reliable method of treating tinnitus compared to that which exists today.

### SUMMARY OF THE PREFERRED EMBODIMENT(S)

A process and system for reducing Tinnitus includes receiving an input audio sound by a user. The user listens to different tones (frequencies) of the received audio to determine both amplitude and frequency of the ringing or tinnitus tone in user's ears and provides tactile response on an input device (keyboard, etc.). The offending frequencies and amplitudes are identified based on the user's responses and a diagnosis from the data collected is produced and a tone map for each ear separately is created and the collection of tones and amplitudes are provided to the user to hear. User is tested for amplitude of these tones and the tones are increased until the audible ringing disappears as the phase of these tones will be shifted from zero to negative 180 degrees and as their level is increased, the ringing will decrease until it is substantially reduced.

### BRIEF DESCRIPTION OF THE DRAWINGS

FIG. 1 is a block diagram of an embodiment of the process of the present invention.

FIG. 2 is a block diagram of the MS response block according to an embodiment of the present invention.

FIG. 3 shows a flow chart of the inventive Max Sound Process according to an embodiment of the present invention.

### DETAILED DESCRIPTION OF THE PREFERRED EMBODIMENT(S)

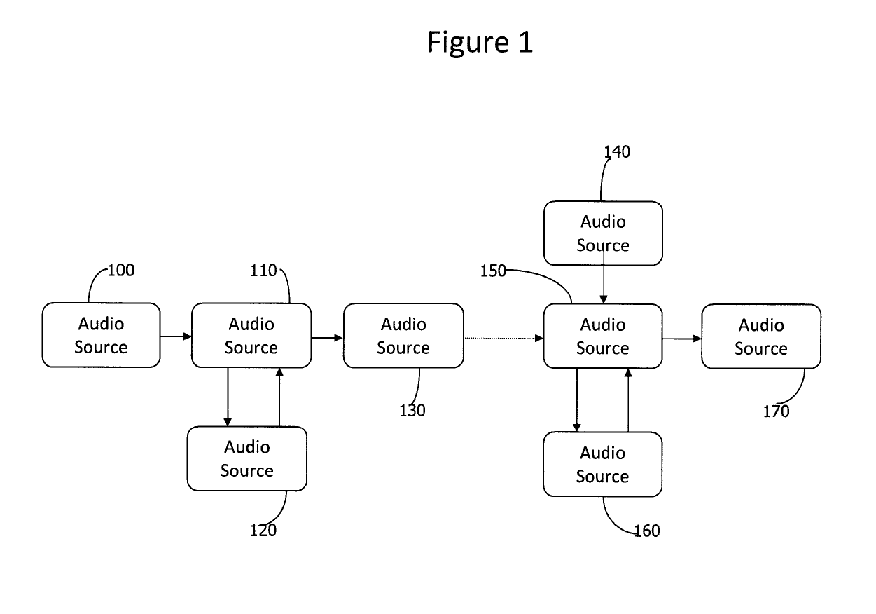

Preferred embodiments of the present invention will now be discussed by reference to the drawings. The MS process for Tinnitus can be either a self contained system or a software package. Both are software programs, but one is built into a dedicated hardware system. FIG. 1 is a representation of a typical use.

Audio Source 100 is preferably a tone generator picked up by the microphone on the earpiece device. In User Response 120 the user will listen to different tones (frequencies) to determine both amplitude and frequency of the ringing or tinnitus tone in their ears. It is important to get both parameters for this to work. The user will provide tactile response on an input device (keyboard, etc.). MS Tone Map 130 will use the user response to identify the offending frequencies and amplitudes of same. This collection of tones and amplitudes will be produced for the user to hear via Collection Device 110. Upon hearing these tones, the user will be tested multiple times for amplitude of these tones. The test will be to increase the tone until the audible ringing disappears. The phase of these tones will be shifted from zero to negative 180 degrees and as their level is increased, the ringing will decrease until it is almost gone. The process will produce a diagnosis from the data collected and produce a tone map, for each ear separately.

As shown by the dotted lines in FIG. 1, Tone Map 130 will be loaded into the Earpiece Device 150 that user is to wear. Tone Map 130 is loaded into a portable tone generating device for the user to carry with them and listen to. If the user is in a quite environment, they should not hear any ringing when the portable device is in use. It will be necessary to test at least every 30 days to adjust for different frequencies and amplitude adjustments until the ringing is gone when not using the portable device. As time progresses, your brain will be re-trained to ignore these harmonics and the sound will virtually disappear.

Earpiece Device 150 is the playback system part of the Tinnitus process. Preferably it will be a dual ear piece with either a wired or wireless connection between them. There could be a separate controller unit (wired or wireless). In one embodiment Ear Piece includes a microphone, digital processor, amplifier, and transducer/speaker (plus the housing for the complete unit). Processor stores the information collected from the testing unit that will make up the tone generation tables with frequency and amplitude. These tables will be turned into audible tones and sent to the amplifier and speaker. There will be a master volume control for the pair of earpieces located on one unit.

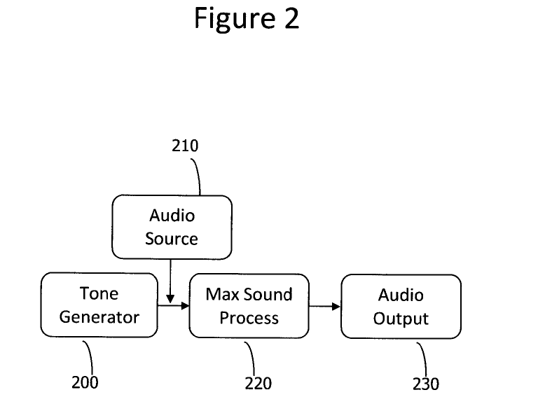

Earpiece Device 150 includes is the MS Response block 160. FIG. 2 shows a description of the block 160. Audio Source 210 (from a microphone on the earpiece) will be summed with the output of the Tone Generator 200 (FIG. 2) in the MS Response section of the earpiece. This process can operate in either stereo or mono mode depending on the type of system the user will want, stereo or mono. The process will be used to add clear and precise harmonic and dynamic content allowing the system to function at a higher level of accuracy. Because the process is fully dynamic, there will also be a reduction in phase anomalies caused by the input audio and/or the earpiece device. While the user is using the earpiece device, it will be generating the tones in the Tone Map. These tones will mix with an audio source 140 that the earpiece collects and transmitted to user's ear 170.

Tone Generator 200 is described next by reference to FIG. 2. In this part of the process, additive synthesis will be used to create the tones used for the “tone Map”. Additive synthesis is a technique of audio synthesis which creates musical timbre. The timbre of an instrument is composed of multiple harmonic or non-harmonic partials (individual sine waves), of different frequencies and amplitudes, that change over time. Additive synthesis allows the emulation of sounds by giving control over the frequency and amplitude of each individual harmonic or partial. Often, each harmonic generator has its own customizable volume envelope, creating a realistic, dynamic sound that changes over time¹². ¹²http://www.princeton.edu/˜achaney/tmve/wiki100k/docs/Additive synthesis.html

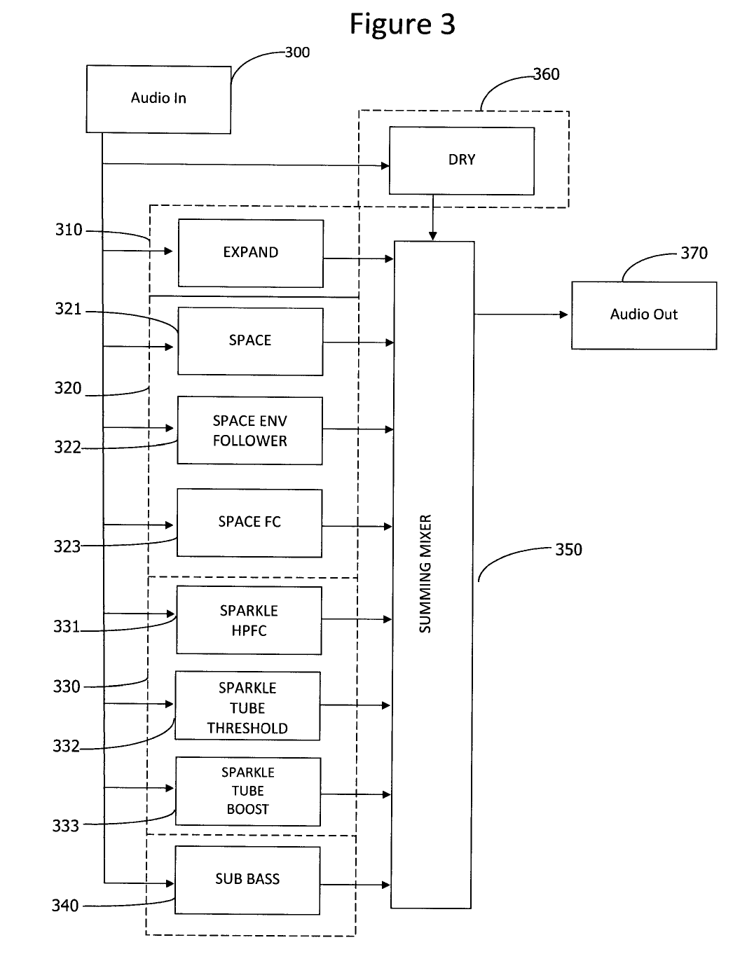

Max Sound Process is explained by reference to FIG. 3. The original audio signal is processed by EXPAND 310, a low pass filter and is then fed to SPACE 320. FIG. 2 depicts three blocks for SPACE 320. The top block SPACE 321 is the output level for this block. The next block 322 is the envelope follower modulation amount, and the last block 323 is the frequency range for SPACE block. The SPACE is an envelope controlled bandpass filter. The output amplitude for space can be set from about 0 to 3, such as about 1.8. The frequency range for SPACE can be about 1000 to about 8000 Hertz. The settings for SPACE can also be preset.

The original audio signal also processed by SPARKLE 330. FIG. 3 depicts three blocks for SPARKLE 330. The top block 331 is the output level for this block, the SPARKLE HPFC 332 set HP filter frequency, and SPARKLE TUBE BOOST 333 sets amount of tube simulator sound. SPARKLE 330 is a high pass filter. The frequency for the high pass filter can be about 4000 to about 10000 Hertz. The tube simulator can be set in single digits from 1-5. The threshold can range from 0-1 in 0.1 digits. The settings for SPARKLE can also be preset.

The original audio signal is processed by SUB BASS 340 in FIG. 2, which adds an amount of dynamic synthesized sub bass to the audio. The frequency of the subbass can be about 120 Hz to less. The four treated audio signals (EXPAND, SPACE, SPARKLE, SUB BASS) are then combined in a summing mixer with DRY 360, which is the original audio input into the software application to produce an audio signal with improved quality.

In more detail, EXPAND 310 is a 4 pole digital low pass filter with an envelope follower for dynamic offset (FIXED ENVELOPE FOLLOWER). This allows the output of the filter to be dynamically controlled so that the output level is equal to whatever the input is to this filter section. (Ex.) If the level at the input is −6dB, then the output will match that. Whenever there is a change at the input, the same change will occur at the output regardless of either positive or negative amounts. The frequency for this filter is 20 to 20 k hertz, in other words it is full range. The intention of this filter is to “warm up” or provide a fuller sound as audio that passes through it. The original sound passes through, and is added to the effected sound for its output. As the input amount increases or decreases (varies), so does the phase of this section. This applies to ALL FILTERS used in this software application. All filters are preferably of the Butterworth type.

SPACE 320 includes several components. They are: SPACE 321—this amount is after the envelope follower and sets the final level of this module. This is the effected signal only, without the original. SPACE ENV FOLLOWER 322—tracks the input amount and forces the output level of this section to match and SPACE FC 323, which sets the center frequency of the 4 pole digital high pass filter used in this section. This filter also changes phase as does the EXPAND one. See reference page.

SPACE blocks 220 are followed by the SPARKLE 230 blocks. Like SPACE 220, there are several components to SPARKLE. SPARKLE HPFC 231 is a 2 pole high pass filter with a preboost which sets the lower frequency limit of this filter. Anything above this setting passes through the filter while anything below is discarded or stopped from passing. SPARKLE TUBE THRESH 232 sets the lower level at which the tube simulator begins working. As the input increases, so does the amount of the tube sound. The tube sound adds harmonics, compression and a slight bit of distortion to the input audio 200. This amount increases slightly as the input level increases. SPARKLE TUBE BOOST 233 sets the final level of the output of this module. This is the effected signal only, without the original.

Next, the SUB BASS 240 module is discussed. This module takes the input signal and uses a low pass filter to set the upper frequency limit to about 100 Hz. An octave divider occurs in the software that changes the input signal to lower by an octave (12 semi tones) and output to the only control in the interface, which is the level or the final amount. This is the effected signal only, without the original.

Outputs from the above modules 210 to 240 are directed into SUMMING MIXER 250 which combines the audio. The levels going into the summing mixer 250 are controlled by the various outputs of the modules listed above. As they all combine with the original signal 200 fed through the DRY 260 module there is interaction in phase, time and frequencies that occur dynamically. These changes all combine to create a very pleasing audio experience for the listener in the form of “enhanced” audio content. For example, a change in a single module can have a great affect on what happens in relation to the other modules final sound or the final harmonic output of the entire software application.

Audio Output—This block passes the audio from the Earpiece Device into the users' ear canal. The end result is a much clearer auditory experience with less “ringing” from Tinnitus.

The system needs all of these parts in order to function correctly. In a typical conventional system for addressing tinnitus, the entire operation would be static in nature therefore being much less efficient because it doesn't have the ability to operate in a dynamic nature to accommodate any changing conditions in a user's environment. Earpieces also have phase anomalies because of their construction. Because the present invention is based on a dynamic process, it greatly lessens that effect on what the user hears, both from Tinnitus and the outside audio user may be listening to. Be it voice, music, environmental, etc.

What is claimed is:

1. A process and system for reducing Tinnitus comprising:
Receiving an input audio sound by a user;
The user listening to different tones (frequencies) of the received audio to determine both amplitude and frequency of the ringing or tinnitus tone in user's ears;
User providing tactile response on an input device (keyboard, etc.);
Identifying the offending frequencies and amplitudes based on the user's response;
Producing a diagnosis from the data collected;
Creating a tone map for each ear separately;
Providing the collection of tones and amplitudes to the user to hear;
Testing user for amplitude of these tones;
Increasing the tones until the audible ringing disappears as the phase of these tones will be shifted from zero to negative 180 degrees and as their level is increased, the ringing will decrease until it is substantially reduced.

2. The process of claim 1 wherein the audio source is a tone generator.

3. The process of claim 2 further comprising:
Receiving an input audio source by an earpiece;
Providing a tone by a tone generator;
Processing the combined tone generator and received input audio by a sound enhancement module;
Transmitting the enhanced sound to the earpiece;
Providing the enhanced sound received by the earpiece to user.

4. The process of claim 1, wherein the enhancement includes the parallel processing the input audio as follows:
A module that is a low pass filter with dynamic offset;
An envelope controlled bandpass filter;
A high pass filter;
Adding an amount of dynamic synthesized sub bass to the audio;
Combining the four treated audio signals in a summing mixer with the original audio.
